(19) United States
(12) Patent Application Publication
(10) Pub. No.: US 2023/0165753 A1
(43) Pub. Date: Jun. 1, 2023

(54) METHOD FOR OPERATING MASSAGE APPARATUS COMPRISING UV STERILIZATION MODULE

(71) Applicant: BODYFRIEND CO., LTD., Seoul (KR)

(72) Inventors: Ju Song HUR, Seoul (KR); Young Ju SONG, Seoul (KR); Sang Joon AN, Seoul (KR)

(21) Appl. No.: 17/921,045

(22) PCT Filed: Apr. 8, 2021
(86) PCT No.: PCT/KR2021/004425

§ 371 Date: Oct. 24, 2022

(30) Foreign Application Priority Data
Apr. 23, 2020 (KR) 10-2020-0049440
Feb. 18, 2021 (KR) 10-2021-0022093
Mar. 5, 2021 (KR) 10-2021-0029653

(51) Int. Cl. A61H 37/00; A61H 9/00; A61L 2/10; A61L 2/24

(52) U.S. Cl. CPC A61H 37/00; A61H 9/0078; A61L 2/10; A61L 2/24; A61H 2201/0149

(57) ABSTRACT

The present disclosure relates to a massage apparatus comprising a UV sterilization module, the massage apparatus comprising: a main frame which forms a framework of the massage apparatus and includes an upper frame and a base frame; a seat pad or a back pad formed on the upper frame, for improving the sitting comfort of a user; arm massage portions formed at opposite sides of the main frame and massaging the arms of the user; the UV sterilization module for sterilizing and disinfecting at least a part of the seat pad, the back pad, or the arm massage portions; a controller for controlling operations of the massage apparatus; and at least one human body sensor which executes a first confirmation for determining whether the user is adjacent to the massage apparatus, wherein the controller, when the user is not adjacent to the massage apparatus, sterilizes the massage apparatus for a preset time period by using the UV sterilization module.

### TECHNICAL FIELD

The present disclosure relates to a massage apparatus including an ultraviolet sterilization module and an operation method thereof.

### BACKGROUND

Massage is a medical auxiliary therapy for controlling modulation of the subject's body, helping blood circulation, and relieving fatigue of the subject by applying various types of mechanical stimulation to a part of the subject's body, such as kneading, pressing, pulling, tapping, or moving a part of the subject's body.

An increase in demand for massage causes an increase in demand for massage apparatuses or massage devices that provide artificial massage functions due to economic circumstances and time reasons. In other words, as the demand for relieving fatigue or stress while relaxing muscles through massage increases, various massage apparatuses using a time and cost effective method have been released. Any type of instrument, device, or apparatus that performs massage through a mechanical device without a separate masseur is referred to as a massage apparatus.

Recently, public interest in sterilization has increased, and there are increasing cases in which each home is equipped with an air purifier or waterpurifier. As a method of purifying air or water by an air purifier or water purifier, a method of filtering foreign substances using a filter, a method of purifying foreign substances using chemicals, or a method of purifying foreign substances using light such as ultraviolet light are used. In addition, due to activation of sharing economy, there are cases in which sucha sterilization module is provided by default in a product.

### SUMMARY

### Technical Problem to be Solved

With recent activation of sharing economy, the number of users sharing one massage device is increasing. in addition, as a massage apparatus gains popularity, there are increasing cases of experiencing displayed massage apparatuses before purchasing it. However, if the massage apparatus used by several people is not sterilized, there are problems in that infectious diseases are transmitted between people, and body odor of a previous user causes discomfort to the user.

### Technical Solution

According to an embodiment of the present disclosure, a massage apparatus includes a main frame defining a frame of the massage apparatus and including an upper frame and a base frame, a seat pad or a back pad formed on the upper frame and increasing a seating feel of a user, an arm massage part formed on both sides of the main frame and providing massage to an arm of the user, an ultraviolet sterilization module for sterilizing and disinfecting at least some of the seat pad, the back pad, or the arm massage part, a controller for controlling an operation of the massage apparatus, and at least one body sensor for performing a first check to determine whether the user is close to the massage apparatus, wherein, when the user is not close to the massage apparatus, the controller sterilizes the massage apparatus for a predetermined time using the ultraviolet sterilization module.

According to an embodiment of the present disclosure, the ultraviolet sterilization module of the massage apparatus may include a light source cover for transmitting ultraviolet light and preventing a foreign substance from entering an ultraviolet light source, an ultraviolet light PCB including the ultraviolet light source and a circuit for driving the ultraviolet light source, a cooler for cooling the ultraviolet light PCB, and a bracket for coupling the ultraviolet light PCB and the cooler to a housing of the massage apparatus.

According to an embodiment of the present disclosure, the light source cover of the massage apparatus may include quartz for transmitting ultraviolet light having a wavelength of 100 nm or more and 280 nm or less.

According to an embodiment of the present disclosure, the housing of the massage apparatus may include a concave portion curved into the housing of the massage apparatus at a position of the ultraviolet sterilization module, the concave portion may include hole to allow the ultraviolet light source located inside the housing of the massage apparatus to emit ultraviolet light to an outside, and a range of sterilization by ultraviolet light emitted from the ultraviolet light source may be determined based on a shape of the concave portion.

According to an embodiment of the present disclosure, while the massage apparatus is sterilized for a predetermined time using the ultraviolet sterilization module, a signal indicating that the user is prevented from approaching a vicinity of the massage apparatus may be output through an output interface.

According to an embodiment of the present disclosure, while the massage apparatus is sterilized for a predetermined time using the ultraviolet sterilization module, the controller may perform a third check to determine whether the user is close to the massage apparatus using the at least one body sensor, and when the user is close to the massage apparatus in the third check, the controller may control and turn off the ultraviolet sterilization module.

According to an embodiment of the present disclosure, a target surface of the massage apparatus, sterilized by the ultraviolet sterilization module, may be located at a position at which the user and the massage apparatus are in contact with each other.

According to an embodiment of the present disclosure, the ultraviolet sterilization module of the massage apparatus may be located inside a housing surrounding an upper end of the upper frame and may emit ultraviolet light to the target surface including the seat pad or the back pad.

According to an embodiment of the present disclosure, the ultraviolet sterilization module of the massage apparatus may be located inside a housing of the arm massage part, may be disposed in front of a hand/arm accommodation space of the housing of the arm massage part, and may emit ultraviolet light toward the hand/arm accommodation space from a front of the hand/arm accommodation space.

According to an embodiment of the present disclosure, the massage apparatus may further include a leg massage part formed in front of the main frame and providing massage to a leg of the user, wherein the ultraviolet sterilization module may be located inside a housing of the leg massage part, may be disposed in front of a foot accommodation space of the housing of the leg massage part, and may emit ultraviolet light toward the foot accommodation space from a front of the foot accommodation space.

According to an embodiment of the present disclosure, the controller of the massage apparatus may estimate a position of a shoulder of a previous user using the massage module, and determine the predetermined time based on the estimated position of the shoulder and a pre-stored table.

According to an embodiment of the present disclosure, the controller of the massage apparatus may determine the predetermined time based on a usage time, a sitting time, or a time of receiving massage of the massage apparatus for a previous user.

In addition, a program for implementing an operation method of the above-described massage apparatus may be recorded on a computer-readable recording medium.

### Advantageous Effect

The massage apparatus according to the present disclosure may be sterilized periodically or s d after a user uses the massage apparatus to prevent bacteria or viruses from raining in the massage apparatus. In addition, it may be possible to prevent odor of existing users from remaining in the massage apparatus. Therefore, a next user may always use the massage apparatus comfortably.

### BRIEF DESCRIPTION OF DRAWINGS

FIG. 1 is a diagram for explaining a massage apparatus according to an embodiment of the present disclosure.

FIG. 2 is a diagram for explaining a main frame according to an embodiment of the present disclosure.

FIG. 3 is a diagram showing a massage apparatus according to an embodiment of the present disclosure.

FIG. 4 is a diagram showing an external device for communicating with a massage apparatus according to an embodiment of the present disclosure.

FIG. 5 is a flowchart showing an operation of a massage apparatus according to an embodiment of the present disclosure.

FIG. 6 is a diagram showing a state of a massage apparatus according to an embodiment of the present disclosure.

FIG. 7 is a diagram showing an ultraviolet sterilization module according to an embodiment of the present disclosure.

FIG. 8 is a diagram showing an ultraviolet sterilization module according to an embodiment of the present disclosure.

FIG. 9 is a flowchart for explaining an operation of a massage apparatus according to an embodiment of the present disclosure.

FIG. 10 is a diagram for explaining a sterilization range of a massage apparatus according to an embodiment of the present disclosure.

FIG. 11 is a diagram for explaining a sterilization range of a massage apparatus according to an embodiment of the present disclosure.

FIG. 12 is a diagram for explaining a method of determining a sterilization time according to an embodiment of the present disclosure.

### BEST MODE

A massage apparatus including a main frame defining a frame of the massage apparatus and including an upper frame and a base frame; a seat pad or a back pad formed on the upper frame and increasing a seating feel f a user; an arm massage part formed on both sides of the main frame and providing massage to n arm of the user; an ultraviolet sterilization module for sterilizing and disinfecting at least some of the seat pad, the back pad, or the arm massage part; a controller for controlling an operation of the massage apparatus; and at least one body sensor for performing a first check to determine whether the riser is close to the massage apparatus, wherein, when the user is not close to the massage apparatus, the controller sterilizes the massage apparatus for a predetermined time using the ultraviolet sterilization module.

### Mode for Disclosure

The present disclosure will now be described more fully with reference to the accompanying drawings, in which exemplary embodiments of the present disclosure are shown. The present disclosure may, however, be embodied in many different forms and should not be construed as being limited to the embodiments set forth herein; rather, these embodiments are provided so that this disclosure will be thorough and complete, and will fully convey the concept of the present disclosure to those skilled in the art.

The terms used in this specification will be briefly described, and the disclosed embodiments will be described in detail.

Although the terms used in the present disclosure are selected from generally known and used terms, terms used herein may be varied depending on operator's intention or customs in the art, appearance of new technology, or the like. In addition, some of the terms mentioned in the description of the present disclosure have been selected by the applicant at his or her discretion, the detailed meanings of which are described in relevant parts of the description herein. Furthermore, it is required that the present disclosure is understood, not simply by the actual terms used but by the meanings of each term lying within.

Expressions in the singular number in this specification include plural expressions unless clearly specified as singular in context. Also, plural expressions include singular expressions unless the context clearly specifies that they are plural.

In the specification, when a certain part is said to “include” a certain component, this indicates that the part may further include another component, rather than necessarily excluding the other component, unless there is a disclosure to the contrary.

The term “part” used in the specification means a software or hardware component, and the “part” performs certain roles. However, the “part” is not meant to be limited to software or hardware. The “part” may be present in an addressable storage medium and may be configured to reproduce one or more processors. Thus, for example, the “part” refers to components such as software components, object-oriented software components, class components and task components, processes, functions, properties, procedures, subroutines, segments of program code, drivers, firmware, microcode, circuitry, data, databases, data structures, tables, arrays, and variables. Functions provided within components and “parts” may be combined into fewer components and “parts” or further separated into additional components and “parts”.

According to an embodiment of the present disclosure, the “part” may be implemented as a processor and a memory. The term “processor” needs to be interpreted broadly to include general-purpose processors, central processing units (CPUs), microprocessors, digital signal processors (DSPs), controllers, microcontrollers, state machines, and the like. In some circumstances, the “processor” may refer to an application specific integrated circuit (ASIC), programmable logic device (PLD), field programmable gate array (FPGA), or the like. The term “processor” refers to a combination of processing devices, such as a combination of a DSP and a microprocessor, a combination of a plurality of microprocessors, a combination of one or more microprocessors combined with a DSP core, or a combination of any other such configurations.

The term “memory” needs to be interpreted broadly to include any electronic component for storing electronic information. The term “memory” refers to various types of processor-readable media such as random access memory (RAM), read-only memory (ROM), non-volatile random access memory (NVRAM), programmable read-only memory (PROM), erasable-programmable read-only memory (EPROM), electrically erasable programmable read-only memory (EEPROM), flash memory, magnetic or optical data storages, registers, and the like. The memory may be said to be in an electronic communication state with the processor as long as a processor is capable of reading information from and/or write information to the memory. A memory integrated with the processor is in an electronic communication state with the processor.

In this specification, an actuator means a component for providing a driving force. For example, the actuator may include a motor, a linear motor, an electric motor, a DC motor, an AC motor, a linear actuator, an electric actuator, and the like, but is not limited thereto.

In this specification, according to an embodiment, a massage apparatus may refer to a massage apparatus including a body massage part and a leg massage part. According to another embodiment, a body massage part 2100 and a leg massage part 2300 may be present as separate apparatuses (e.g., a body massage apparatus and a leg massage apparatus), and the massage apparatus may refer to a body massage apparatus or a leg massage apparatus.

Exemplary embodiments of the present disclosure are described in detail so as for those of ordinary skill in the art to easily implement the present disclosure with reference to the accompanying drawings. To clearly describe the present disclosure, a part without concerning to the description is omitted in the drawings.

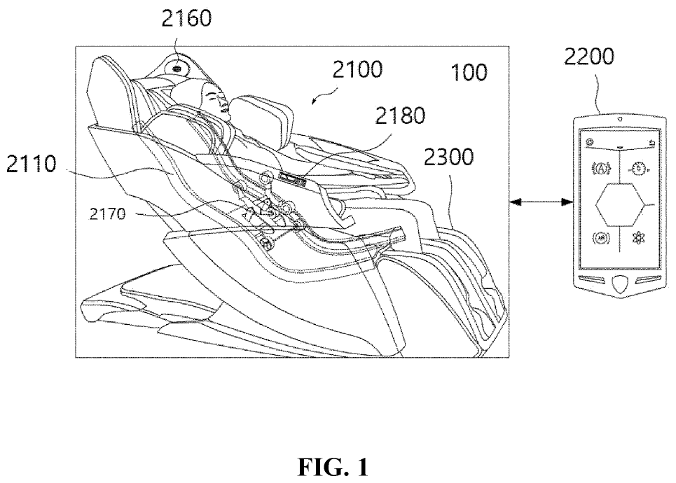

FIG. 1 is a diagram for explaining a massage apparatus according to an embodiment of the present disclosure.

A massage apparatus 100 according to an embodiment of the present disclosure may define a region for accommodating at least a part of a body of a user, and include the body massage part 2100 for massaging a torso of the user, and the leg massage part 2300 for massaging a leg of the user.

The body massage part 2100 may provide massage to at least a part of the body of the user. The body massage part 2100 may include a massage module 2170 for providing a massage function to at least a part of the body of the user, an audio output module 2160 for providing an arbitrary form of audio output to the user, a main frame 2110 defining a frame of the body massage part 2100, and a user input interface 2180 for receiving an arbitrary type of input from the user.

Components included in the aforementioned body massage part 2100 are merely exemplary embodiments, and the body massage part 2100 may include various components other than the aforementioned components.

The shape and structure of the massage apparatus 100 shown in FIG. 1 are only exemplary, and the massage apparatus 100 in various forms is also included in the scope of the present disclosure unless departing from the scope defined by the claims of the present disclosure.

The body massage part 2100 may define any type of space for accommodating a user.

The body massage part 2100 may have a space corresponding to the shape of the body of the user. For example, as shown in FIG. 1, the body massage part 2100 may be implemented as a seating type for accommodating the entire or partial body of the user.

A portion of the body massage part 2100, which is in contact with the ground, may include any material for increasing frictional force or any member (e.g., non-slip pad) for increasing frictional force, and include a wheel for enhancing the mobility of the massage apparatus 100.

At least a part of the body massage part 2100 may slide. For example, when the body massage part 2100 starts massage, at least a part of the body massage part 2100 may slide forward. The body massage part 2100 may be tilted backward. As a result, the body massage part 2100 may provide massage in a backward tilted state.

According to an embodiment of the present disclosure, the massage apparatus 100 may include at least one air cell (not shown). The air cell may be located in a shoulder part and pelvis part of the user, an arm massage part 2140, the leg massage part 2300, or the like, but is not limited thereto and may be placed in various parts of the massage apparatus 100.

The massage apparatus 100 may include an air supply, and the air supply may inflate the air cell by supplying air to the air cell. The air supply may be located inside the body massage part 2100 and may be located in the leg massage part 2300. The air supply may be located outside the massage apparatus 100.

The massage apparatus 100 may include the leg massage part 2300. The leg massage part 2300 may be formed in front of the main frame 2110 to provide massage to legs of the user.

The leg massage part 2300 may provide leg massage to the user. For example, the leg massage part 2300 may include a calf massage part for massaging a calf of the user and/or a foot massage part for massaging feet of the user.

The leg massage part 2300 may be adjustable in length according to the physical characteristics of the user. For example, when a tall user uses the massage apparatus 100, the leg massage part 2300 needs to be long because the length of the calf is long. In addition, when a short user uses the massage apparatus 100, the leg massage part 2300 needs to be shortened because the length of the calf is short. Accordingly, the leg massage part 2300 may provide leg massage customized for the height of the user.

The massage module 2170 may be provided inside the body massage part 2100 to provide any type of mechanical stimulation to a user accommodated in the body massage part 2100. As shown in FIG. 1, the massage module 2170 may move along the main frame 2110 provided inside the body massage part 2100.

For example, the main frame 2110 of the body massage part 2100 may be equipped with a rack gear, and the massage module 2170 may provide mechanical stimulation to various parts of the body of the user while moving along the rack gear. The massage module 2170 may include a ball massage part or a roller massage part, but is not limited thereto.

The main frame 2110 may define a frame of an internal structure of the body massage part 2100, and may be implemented with a metal material or a plastic material. For example, the main frame 2110 may be implemented with iron, alloy, steel, etc., but is not limited thereto and may be implemented with various hard materials.

The massage apparatus 100 according to an embodiment of the present disclosure may include the arm massage part 2140. The arm massage part 2140 may be formed on both sides of the main frame 2110 and may provide massage to arms of the user.

The massage apparatus 100 according to an embodiment of the present disclosure may include the audio output module 2160. The audio output module 2160 may be provided at various positions. For example, the audio output module 2160 may include a plurality of output interfaces such as an upper audio output interface disposed on an upper part of a seat part in contact with the user, a front audio output interface attached to a front end of the arm massage part 2140 at each of right and left sides of the seat part, and/or a rear audio output interface attached to a rear end of the arm massage part, but is not limited thereto. In this case, the audio output module 2160 may provide 3D sound such as 5.1 channels, but is not limited thereto.

According to an embodiment of the present disclosure, the user may control the massage apparatus 100 using a massage apparatus control device 2200. The massage apparatus control device 2200 may be connected to the massage apparatus 100 through wired communication and/or wireless communication.

The massage apparatus control device 2200 may include a remote controller, a cellular phone, and Personal Digital Assistant (PDA), but is not limited thereto and may include various electronic devices to be connected to the massage apparatus 100 through wired or wireless communication.

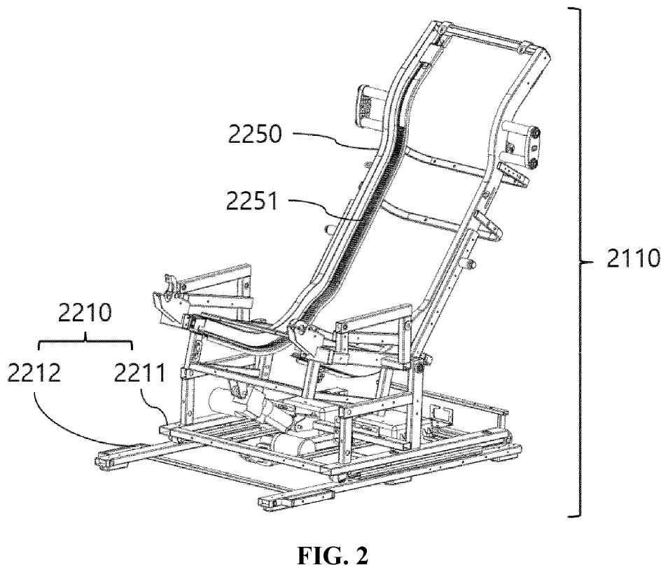

FIG. 2 is a diagram for explaining a main frame according to an embodiment of the present disclosure.

The main frame 2110 according to an embodiment of the present disclosure may include an upper frame 2250 including the massage module 2170 and a base frame 2210 for supporting the upper frame 2250.

A rack gear 2251 may be provided on at least a part of the upper frame 2250. The rack gear 2251 may be a member for guiding vertical movement of the body massage module 2170, and include a plurality of valleys and a plurality of crests.

According to an embodiment of the present disclosure, the rack gear 2251 may be provided on both sides of the upper frame 2250 to face each other, and the body massage module 2170 may move along the rack gear 2251.

In FIG. 2, the rack gear 2251 may be formed in an up-down direction, but is not limited thereto. The rack gear 2251 may include a forward-backward rack gear and an up-down rack gear. In the present disclosure, the forward-backward direction means a direction from the body massage module 2170 to the user or from the user to the body massage module 2170, and may also be referred to as the Z-axis direction.

The body massage module 2170 may include a gear engaged with the rack gear 2251. In more detail, the body massage module 2170 may include gears that are engaged with the forward-backward rack gear and the up-down rack gear. As the gear rotates by the actuator provided in the body massage module 2170, the body massage module 2170 may move forward, backward, upward, or downward.

As the body massage module 2170 moves forward, the intensity of massage may increase. As the body massage module 2170 moves backward, the intensity of massage may decrease.

The rack gear 2251 may be implemented with a metal material or a plastic material. For example, the rack gear 2251 may be implemented with iron, steel, alloy, reinforced plastic, melamine resin, phenol resin, etc., but is not limited thereto.

The upper frame 2250 may be implemented in various shapes. For example, the upper frame 2250 may be classified into an S frame, an L frame, an S&L frame, and a double S&L frame depending on the shape, but is not limited thereto.

The S frame refers to a frame in which at least a part of the upper frame 2250 includes a curved shape like “S”. The L frame means a frame in which at least a part of the upper frame 2250 includes a curved shape like “L”, and the S&L frame means a frame including both a curved shape like “S” and a curved shape like “L”. The double S&L frame means a frame that includes a curved shape like an “L” and a curved shape like an “S” of two parts.

A seat pad or a back pad may be formed on the upper frame 2250 to increase a seating feel of the user. The seat pad or the back pad may include an elastic material. Therefore, the user may feel comfortable when sitting on the massage apparatus. In addition, an outer surface of the seat pad or the back pad may include a fabric or leather material designed for aesthetics.

The base frame 2210 refers to a part of the main frame 2110, which supports the upper frame 2250 and is in contact with the ground. The base frame 2210 may include a base upper frame 2211 and a base lower frame 2212.

The base upper frame 2211 may support the upper frame 2250, and the base lower frame 2212 may be in contact with the ground. The base upper frame 2211 may be located to be in contact with the base lower frame 2212.

According to an embodiment of the present disclosure, the base upper frame 2211 may move along the base lower frame 2212. For example, the base upper frame 2211 may slide forward or backward along the base lower frame 2212. In this case, the upper frame 2250 may be connected to the base upper frame 2211 and may move along with movement of the base upper frame 2211.

For example, when the base upper frame 2211 moves forward, the upper frame 2250 may also move forward, and when the base upper frame 2211 moves backward, the upper frame 2250 may also move backward. Accordingly, sliding movement of the body massage part 2100 may be allowed.

In detail, to allow movement of the base upper frame 2211, a moving wheel may be provided below the base upper frame 2211. In addition, a guide member for guiding the moving wheel may be provided above the base lower frame 2212. The moving wheel provided in the base upper frame 2211 may move along the guide member provided on the base lower frame 2212, and thus forward movement or backward movement of the base upper frame 2211 may be allowed.

According to another embodiment of the present disclosure, the massage apparatus 100 may not provide a sliding function, and in this case, the base frame 2210 may not be separated into upper and lower frames.

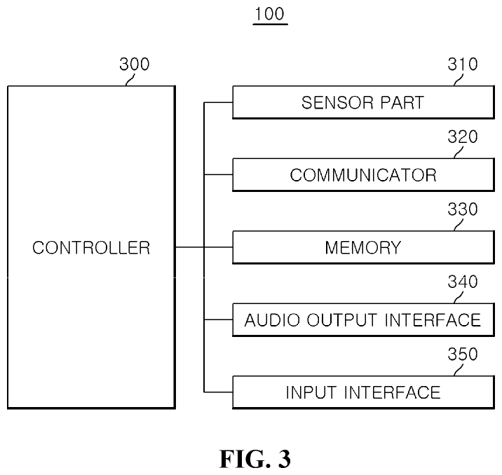

FIG. 3 is a diagram showing a massage apparatus according to an embodiment of the present disclosure.

The massage apparatus 100 may include at least one of a controller 300, a sensor part 310, a communicator 320, a memory 330, an audio output interface 340, or an input interface 350. The controller 300 may include at least one processor and a memory. The at least one processor may execute commands stored in the memory.

The controller 300 may control an operation of the massage apparatus 100. The controller 300 may include one processor or may include a plurality of processors. When the controller 300 includes a plurality of processors, at least some of the plurality of processors may be physically spaced apart from each other. In addition, the massage apparatus 100 is not limited thereto and may be implemented in various ways.

According to an embodiment of the present disclosure, the controller 300 may control an operation of the massage apparatus 100. For example, the massage apparatus 100 may include a plurality of actuators, and the massage apparatus 100 may control an operation of the plurality of actuators, and thus may control an operation of the massage apparatus 100. For example, the massage apparatus 100 may include at least one of a driver for moving an armrest frame support, an actuator for moving the massage module 2170, at least one actuator included in the massage module, a back angle actuator, a leg angle actuator, a foot massage actuator, a leg length control actuator, or a sliding actuator, and the controller 300 may control the operation of the massage apparatus 100 by controlling them.

The actuator for moving the massage module may be an actuator for vertical movement of the massage module 2170, and the massage module 2170 may move along the rack gear by an operation of the actuator for moving the massage module 2170.

The back angle actuator may be an actuator for adjusting an angle of a part of the massage apparatus 100, which is in contact with the back of the user, and a back angle of the massage apparatus 100 may be adjusted by an operation of the back angle actuator.

The leg angle actuator may be an actuator for adjusting an angle of the leg massage part 2300 of the massage apparatus 100, and an angle between the leg massage part 2300 and the body massage part 2100 may be adjusted by an operation of the leg angle actuator.

The foot massage actuator may be an actuator for operating the foot massage module included in the leg massage part 2300. The massage apparatus 100 may provide foot massage to the user using the foot massage actuator.

The massage module 2170 may include at least one actuator, and the controller 300 may provide various massage operations by operating at least one actuator. For example, the controller 300 may operate at least one actuator included in the massage module 2170, and may provide tapping massage, kneading massage, etc. but not limited thereto, and may provide various massage operations.

The leg length control actuator may be an actuator for adjusting the length of the leg massage part 2300. For example, the controller 300 may adjust the length of the leg massage part 2300 according to the user through the leg length control actuator, and as a result, the user may receive massage suitable for a body type.

The sliding actuator may enable a sliding operation of the massage apparatus 100. For example, an upper frame of a horizontal base may move forward or backward by the operation of the sliding actuator, and as a result, the upper frame connected to the upper frame of the horizontal base may also move forward or backward.

The memory 330 may be included in the controller 300 or may be present outside the controller 300. The memory 330 may store various types of information related to the massage apparatus 100. For example, the memory 330 may include massage control information and may include personal authentication information, but is not limited thereto.

The memory 330 may be implemented through a non-volatile storage medium for continuously storing arbitrary data. For example, the memory 330 may include, but is not limited to, storage devices based on a flash memory and/or a battery-backed memory as well as disks, optical disks, and magneto-optical storage devices.

The memory 330 may be a main storage device directly accessed by a processor, such as random access memory (RAM) including dynamic random access memory (DRAM) and static random access memory (SRAM), and refer to a volatile storage device in which stored information is instantly erased when power is turned off, but is not limited thereto. The memory 330 may be operated by the controller 300.

The massage apparatus 100 may include the sensor part 310. The sensor part 310 may acquire various types of information using at least one sensor. The sensor part 310 may be equipped with a sensor using a measuring device such as pressure, potential, and optics. For example, the sensor may include a pressure sensor, an infrared sensor, a touch sensor, and the like, but is not limited thereto.

The sensor part 310 may include a biometric information acquisition sensor. The biometric information acquisition sensor may obtain fingerprint information, face information, voice information, iris information, weight information, electrocardiogram information, body composition information, and the like, but is not limited thereto and may include various types of biometric information.

According to another embodiment of the present disclosure, the massage apparatus 100 may detect a contact area and/or a contact position with a user through sensors. In addition, the massage apparatus 100 may provide customized massage based on information acquired through sensors. The massage apparatus 100 may include a communicator. The communicator of the massage apparatus 100 may receive a signal from an external device. The controller 300 may obtain a result signal by processing the received signal. The communicator may output the result signal to an external device.

The communicator 320 may communicate with a module inside the massage apparatus 100, an external massage apparatus, and/or a user terminal through an arbitrary type of network. The communicator may include a wired/wireless access module for network access. As a wireless access technology, for example, wireless LAN (WLAN) (Wi-Fi), wireless broadband (Wibro), world interoperability for microwave access (Wimax), high speed downlink packet access (HSDPA), and the like may be used. As a wired access technology, for example, digital Subscriber Line (XDSL), Fibers to the home (FTTH), Power Line Communication (PLC), and the like may be used. In addition, a network connector may include a short-distance communication module to transmit and receive data with any device/terminal located in a short distance. For example, Bluetooth, Radio Frequency Identification (RFID), Infrared Data Association (IrDA), Ultra-Wideband (UWB), ZigBee, etc. may be used as short range communication technologies, but the present disclosure is not limited thereto.

The massage apparatus 100 may further include the input interface 350 or an output interface. The massage apparatus 100 may receive input from the user using the input interface 350. The massage apparatus 100 may output a processing result of the controller 300 to the output interface. The input interface 350 and the output interface may be integrated into each other to be implemented as one device. For example, a touchscreen may receive input from the user and simultaneously may output an image.

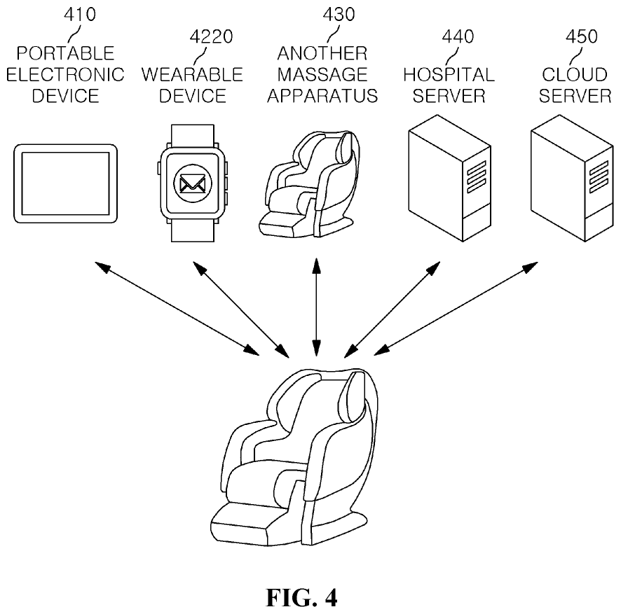

In detail, the input interface 350 may receive a command related to operation control of the massage apparatus 100 from the user, and the input interface 350 may be implemented in various forms. For example, the input interface 350 may be included in the body massage part 2100 and the leg massage part 2300, but is not limited thereto. The input interface 350 may include the user input interface 2180 of FIG. 1, the massage apparatus control device 2200, or various external devices to be described with reference to FIG. 4.

The massage apparatus 100 may acquire various commands from the user through the input interface 350. For example, the massage apparatus 100 may receive an arbitrary command for selection of a massage module, selection of a massage type, selection of massage intensity, selection of a massage time, selection of a massage part, selection of the position and operation of the body massage part 2100, selection for On-Off of power of the massage apparatus 100, selection for whether or not to operate a heating function, and selection for sound reproduction, but is not limited thereto.

The massage apparatus 100 may provide an interface for selection of a massage mode. For example, the input interface 350 or the output interface may include the massage apparatus control device 2200. Through the massage apparatus control device 2200, a medical massage list of various modes related to body improvement may be listed.

The medical massage mode may include at least one of a concentration mode, a meditation mode, a recovery mode, a stretching mode, a sleep mode, a vitality mode, a golf mode, a hip-up mode, a student mode, a zero gravity mode, or growth mode.

According to another embodiment of the present disclosure, the input interface 350 may include hot key type buttons and/or a selection button for executing direction selection, cancellation, and input according to a preset user setting function or a self-preset function.

The input interface 350 may be implemented as a touch screen, a key pad, a dome switch, a touch pad (static pressure/capacity), a jog wheel, a jog switch, and the like, but is not limited thereto. In addition, the input interface 350 may obtain a command through a speech of the user based on voice recognition technology.

According to an embodiment of the present disclosure, the output interface may include a display for displaying an operation state of the massage apparatus 100 or the current state of the user. In this case, the display may include at least one of a liquid crystal display (LCD), a thin film transistor-liquid crystal display (TFT LCD), an organic light-emitting diode (OLED), a flexible display, or a 3D display, but is not limited thereto.

The output interface may include the audio output interface 34. The audio output interface 340 may include the audio output module 2160 of FIG. 1. The audio output interface 340 may provide any type of audio output to the user. For example, the audio output interface 340 may provide brain stimulation to the user by outputting a sound source and/or binaural beats optimized for a massage pattern provided by the massage apparatus 100 to the user. The audio output interface 340 may output a sound signal received through a network (not shown) or stored in an internal/external storage medium (not shown). For example, the audio output interface 340 may output sound according to control of a user terminal through network connection (for example, Bluetooth connection) with the user terminal. In addition, the audio output interface 340 may output any type of sound signal generated in relation to an operation of the massage apparatus 100.

Those skilled in the art will understand that the present disclosure is implemented in combination with other program modules and/or in a combination of hardware and software. For example, the present disclosure may be implemented by a computer-readable medium.

Any medium accessible by a computer may be a computer-readable medium, and the computer-readable medium may include volatile and non-volatile media, transitory and non-transitory media, and removable and non-removable media. As a non-limiting example, computer-readable medium may include computer-readable storage media and computer-readable transmission media.

Computer readable storage media may include volatile and non-volatile media, transitory and non-transitory media, and removable and non-removable media, implemented in any method or technology for storing information such as computer readable instructions, data structures, program modules or other data. The computer-readable storage media may include RAM, ROM, EEPROM, flash memory, other memory technologies, CD-ROM, digital video disk (DVD), other optical disk storage devices, magnetic cassettes, magnetic tapes, magnetic disk storage devices, or any other medium to be accessed by other magnetic storage devices or computers and to be used to store desired information, but is not limited thereto.

FIG. 4 is a diagram showing an external device for communicating with the massage apparatus 100 according to an embodiment of the present disclosure.

The massage apparatus 100 may communicate with an external device by wire or wirelessly and transmit/receive various data.

The external device may include a portable electronic device 410 such as an AI speaker, a tablet, or a smartphone. The portable electronic device 410 may be a dedicated portable electronic device for the massage apparatus 100 or a general-purpose portable electronic device. The external device may include a wearable device 420 such as a smart watch or a smart band. The external device may include another massage apparatus 430 other than the massage apparatus 100 currently used by the user. The external devices may include a hospital server 440. The external device may include a personal health record (PHR) server. The external device may include a cloud server 450. The external device may include a medical measuring device such as an electronic weight scale, a blood glucose meter, or a blood pressure meter.

Although some examples of the external device have been described in the present disclosure, all devices for communicating with the massage apparatus 100 by wired or wirelessly and exchanging information may correspond to the external device.

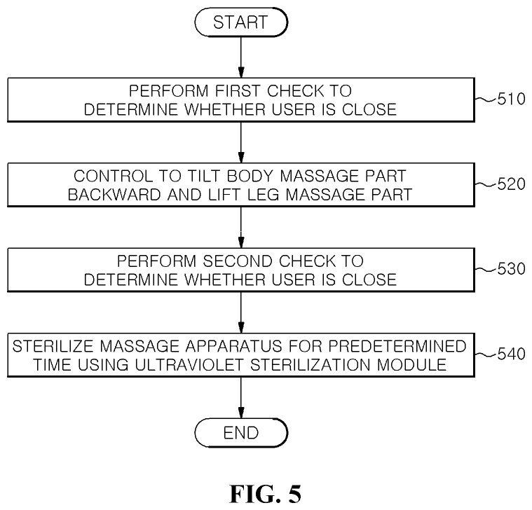

FIG. 5 is a flowchart showing an operation of a massage apparatus according to an embodiment of the present disclosure.

The massage apparatus 100 may include an ultraviolet sterilization module 700. The ultraviolet sterilization module may be a module for sterilizing and disinfecting at least a part of the massage apparatus 100 using ultraviolet light. The ultraviolet sterilization module 700 may be a module for sterilizing and disinfecting at least a part of a seat pad, a back pad, or an arm massage part. The ultraviolet sterilization module 700 may be a module for sterilizing and disinfecting at least a part of the leg massage part. The ultraviolet sterilization module 700 may sterilize a target surface of the massage apparatus 100, which is a contact area between the user and the massage apparatus. The ultraviolet sterilization module 700 may be a module for sterilizing a target surface of the massage apparatus 100, which corresponds to at least one of a neck, a hand, or a foot of the user. Ultraviolet light emitted from the ultraviolet sterilization module 700 may include one of UVA, UVB, and UVC. A configuration of the ultraviolet sterilization module 700 will be described in detail later. Here, the UVA may have a wavelength of 320 nm or more and 400 nm or less. In addition, the UVB may have a wavelength of 280 nm or more and 320 nm or less. In addition, the UVC may have a wavelength of 100 nm or more and 280 nm or less.

The massage apparatus 100 may operate the ultraviolet sterilization module automatically or by user selection. The massage apparatus 100 may operate an ultraviolet sterilization module based on an input of the user. The massage apparatus 100 may automatically operate the ultraviolet sterilization module. For example, the massage apparatus 100 may operate the ultraviolet sterilization module after the user is provided with massage and gest up. Alternatively, the massage apparatus 100 may operate the ultraviolet sterilization module at a predetermined period. The predetermined period may be set by a user or a manufacturer. When operating the ultraviolet sterilization module, the massage apparatus 100 may perform the following operations.

The massage apparatus 100 may perform operation 510 of performing a first check to determine whether a user is close to the massage apparatus using at least one body sensor when the massage mode is terminated. The massage mode may include at least one of a concentration mode, a meditation mode, a recovery mode, a stretching mode, a sleep mode, a vitality mode, a golf mode, a hip-up mode, a student mode, a zero gravity mode, or a growth mode. The massage apparatus 100 may vary the massage pattern according to the massage mode.

The massage mode may correspond to a massage pattern list. Depending on the massage mode, the massage pattern list may be different. Depending on the massage mode, different massage pattern lists may be predetermined. The massage pattern list may include a plurality of massage patterns. The massage pattern list may be a list in which massage patterns to be performed by the massage apparatus 100 are arranged in chronological order. The massage apparatus may perform the massage pattern from the top in the massage pattern list. The massage apparatus may provide massage to the user by sequentially performing a plurality of massage patterns included in the massage pattern list.

When the massage mode ends, this may mean the state in which the massage apparatus 100 does not provide massage to the user. The massage apparatus 100 may determine whether to currently provide massage to the user. When massage is not currently provided to the user, the massage apparatus 100 may perform the first check. The first check may be to determine whether the user is close to the massage apparatus 100 using at least one body sensor.

At least one body sensor may include a seating sensor (e.g., pressure sensor) or a proximity sensor. The massage apparatus 100 may include a seating sensor at a site where the user sits. When the user sits on the massage apparatus 100, a seating sensor may be pressed, and a signal indicating seating of the user may be transmitted to the controller 300. The massage apparatus 100 may determine that the user is sitting on the massage apparatus 100 based on a signal from the seating sensor. That is, the massage apparatus 100 may determine that the user is close to the massage apparatus 100 based on a signal from the seating sensor.

The massage apparatus 100 may include a proximity sensor included in the body sensor. The proximity sensor may include an ultrasonic sensor or an infrared sensor. The proximity sensor may be disposed on an outer surface of the massage apparatus 100. When an object or a user approaches the massage apparatus 100, the proximity sensor may transmit a signal indicating that the object or the user approaches the massage apparatus 100 to the controller 300. The massage apparatus 100 may determine that the user or the object is close to the massage apparatus 100 based on the proximity sensor.

The body sensor may include the massage module 2170. The massage apparatus 100 may determine whether the user is close to the massage apparatus 100 using the massage module 2170. For example, when the massage module 2170 moves up and down, if a load of the driver (actuator) to move the massage module 2170 is equal to or greater than a predetermined threshold value, the massage apparatus 100 may determine that the user is sitting on the massage apparatus 100.

The massage apparatus 100 may determine whether a user is close to the massage apparatus 100 using at least one of a seating sensor, a proximity sensor, or the massage module 2170. According to an embodiment of the present disclosure, the massage apparatus 100 may determine whether the user is close to the massage apparatus 100 using the seating sensor and the massage module 2170 for the first check. However, the massage apparatus 100 is not limited thereto, and may determine whether the user is close to the massage apparatus 100 using all of the seating sensor, the proximity sensor, and the massage module 2170.

If the user is not close to the massage apparatus by the first check, the massage apparatus 100 may perform a control operation 520 of tilting the body massage part 2100 backward and lifting the leg massage part in such a way that the user is not capable of climbing the massage apparatus.

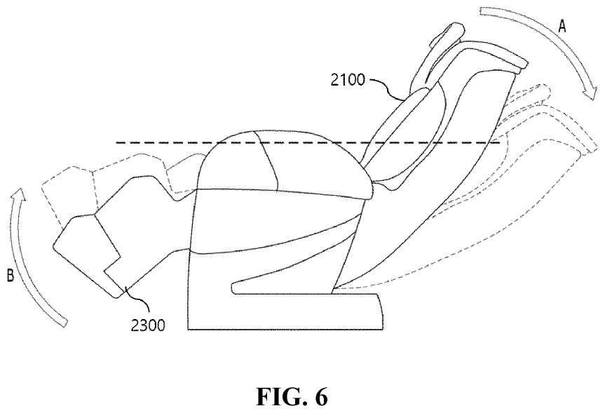

The massage apparatus 100 may promote user safety by allowing the ultraviolet sterilization module to emit ultraviolet light only when the user is not close to the massage apparatus 100. To this end, if the user is not close to the massage apparatus 100 by the first check, the massage apparatus 100 makes it difficult for the user to sit on the massage apparatus 100, thereby preventing the user from climbing the massage apparatus 100 during sterilization. A condition in which it is difficult for the user of the massage apparatus 100 to sit on the massage apparatus 100 will be described in detail together with FIG. 6.

FIG. 6 is a diagram showing a state of a massage apparatus according to an embodiment of the present disclosure.

Initially, the massage apparatus 100 may be in a basic state. The basic state is a posture of the massage apparatus 100 when the massage apparatus 100 does not provide massage, and may be a state in which a user is capable of easily sitting on the massage apparatus 100. The basic state may be a state in which the leg massage part 2300 is substantially perpendicular to the ground. In the basic state, when the user sits on the massage apparatus 100, the knee of the user may be bent, and the calf bone may be in a state almost perpendicular to the ground. The basic state may be a state in which the body massage part 2100 is substantially perpendicular to the ground. In the basic state, when the user sits on the massage apparatus 100, the back of the user may be in a raised state.

The massage apparatus 100 may control the body massage part 2100 to tilt backward as shown in A of FIG. 6 in order to make it difficult for the user to climb the massage apparatus 100. In addition, the massage apparatus 100 may control the leg massage part 2300 to be lifted as shown in B of FIG. 6 in order to make it difficult for the user to climb the massage apparatus 100. The massage apparatus 100 may tilt the body massage part 2100 backward and lift the leg massage part 2300 by controlling an actuator (driver) related to movements of the body massage part 2100 and the leg massage part 2300.

Referring back to FIG. 5, the massage apparatus 100 may perform operation 530 of performing a second check to determine whether the user is close to the massage apparatus using at least one body sensor.

The massage apparatus 100 may determine whether the user is close to the massage apparatus 100 using at least one of a seating sensor, a proximity sensor, or the massage module 2170 while performing the second check.

The second check may be performed in the same way as the first check. According to an embodiment of the present disclosure, the massage apparatus 100 may determine whether the user is close to the massage apparatus 100 using a seating sensor and the massage module 2170 for the second check. However, the present disclosure is not limited thereto, and the massage apparatus 100 may determine whether the user is close to the massage apparatus 100 using all of the seating sensor, the proximity sensor, and the massage module 2170 for the second check.

The second check may be performed in a different way from the first check. For example, the massage apparatus 100 may determine whether the user is close to the massage apparatus 100 using a seating sensor and the massage module 2170 for the first check. In addition, the massage apparatus 100 may determine whether the user is close to the massage apparatus 100 using a proximity sensor for the second check.

The massage apparatus 100 may further promote user safety by performing the second check as well as the first check.

When the use is not close to the massage apparatus in the second check, the massage apparatus 100 may perform operation 540 of sterilizing the massage apparatus for a predetermined time using an ultraviolet sterilization module.

The predetermined time may be determined by a user or a manufacturer. The predetermined time may be 5 minutes to 15 minutes. The massage apparatus 100 may determine a predetermined time based on a sensor signal. For example, the massage apparatus 100 may determine a predetermined time using at least one of a body size of a previous user, temperature, humidity, or dust concentration around the massage apparatus.

A distance between the ultraviolet sterilization module 700 and a target surface of the massage apparatus sterilized by the ultraviolet sterilization module may be 20 cm to 30 cm. If the ultraviolet sterilization module 700 is excessively close to the target surface, a region of the target surface is excessively reduced, and thus an entire desired region may not be sterilized. In addition, if the target surface is excessively far from the ultraviolet sterilization module 700, the intensity of ultraviolet light may decrease and it may take very long for actual sterilization to occur.

While the massage apparatus 100 is sterilized for a predetermined time using the ultraviolet sterilization module 700, the massage apparatus 100 may perform an operation of outputting a signal indicating that the user is prevented from approaching the vicinity of the massage apparatus through the output interface. As described above, the output interface may include a display, an LED, or the audio output interface 340. The massage apparatus 100 may indicate that the massage apparatus 100 is currently being sterilized using sound or light. The user may easily know that the massage apparatus 100 is being sterilized and may not approach the massage apparatus 100.

The massage apparatus 100 may include a sensor for detecting whether ultraviolet light are reflected. For example, the massage apparatus 100 may include an ultraviolet detection sensor. The massage apparatus 100 may be controlled to emit ultraviolet light for a short time by the ultraviolet sterilization module 700. The massage apparatus 100 may determine whether the ultraviolet detection sensor detects ultraviolet light. The massage apparatus 100 may perform ultraviolet sterilization using the ultraviolet sterilization module 700 when the Ultraviolet detection sensor does not detect ultraviolet light.

When the ultraviolet detection sensor detects ultraviolet light, the massage apparatus 100 may output a message indicating to remove a reflector from the target surface. When the ultraviolet detection sensor detects ultraviolet light, the massage apparatus 100 may control the ultraviolet sterilization module 700 to periodically emit ultraviolet light for a short period of time. The massage apparatus 100 may stop an operation of the ultraviolet sterilization module 700 until the ultraviolet detection sensor does not detect ultraviolet light. The massage apparatus 100 according to the present disclosure may prevent ultraviolet light from being emitted to the outside of a surface of the massage apparatus 100 by a reflector.

The massage apparatus 100 may return back to a basic state when sterilization is completed by the ultraviolet sterilization module 700.

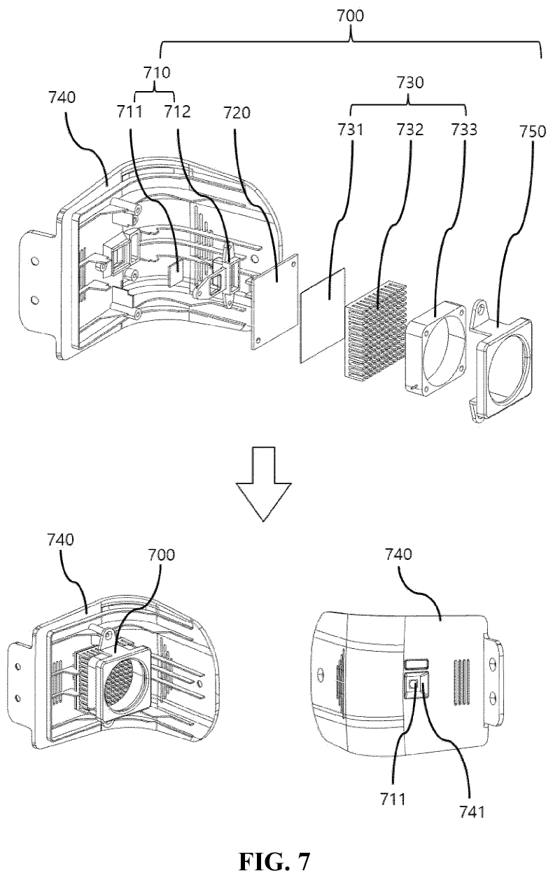

FIG. 7 is a diagram showing an ultraviolet sterilization module according to an embodiment of the present disclosure.

The ultraviolet sterilization module 700 may include a light source cover 710. The light source cover 710 may be a component for transmitting ultraviolet light and preventing foreign substances from entering an ultraviolet light source. The light source cover 710 may include quartz 711 and a light source cover fixer 712. The quartz 711 may be a component for transmitting ultraviolet light emitted from the ultraviolet light source. In detail, the quartz 711 may transmit a wavelength of 100 nm or more and 280 nm or less. The quartz 711 may be a component for preventing foreign substances from entering the ultraviolet light source. The light source cover fixer 712 may be a component for fixing the quartz 711 to a housing 740 of the massage apparatus 100. The light source cover fixer 712 may be a component for fixing the quartz 711 to an ultraviolet light PCB 720. However, the present disclosure is not limited thereto, and the massage apparatus 100 may not include the quartz 711.

The ultraviolet sterilization module 700 may include the ultraviolet light PCB 720. The ultraviolet light PCB 720 may include an ultraviolet light source and a circuit for driving the ultraviolet light source. The ultraviolet light source may emit at least one of UVA, UVB, or UVC.

The ultraviolet light source may include an LED or a mercury lamp.

The ultraviolet sterilization module 700 may include a cooler 730 for cooling the ultraviolet light PCB 720. Heat may be emitted from a light source of the ultraviolet light PCB 720 or a power source for driving the light source. In addition, when the ultraviolet light PCB 720 is maintained at a high temperature, the lifespan of the ultraviolet sterilization module 700 may decrease, and thus cooling may be required. Accordingly, the ultraviolet sterilization module 700 may include a cooler 730.

The cooler 730 may include a thermal tape 731. The thermal tape 731 may be a component for coupling the ultraviolet light PCB 720 and a heat sink 732. In addition, the thermal tape 731 may have high thermal conductivity to transfer heat emitted from the ultraviolet light

PCB 720 to the heat sink 732.

The cooler 730 may include the heat sink 732. The heat sink 732 may be made of metal. The heat sink 732 may include a plurality of cooling plates. Heat transferred from the ultraviolet light PCB 720 may be cooled by cold air flowing between cooling plates of the heat sink 732.

The cooler 730 may include a fan 733. The fan 733 may lower the temperature of the ultraviolet light PCB 720 by blowing cool air into the heat sink 732 or discharging hot air from the heat sink 732.

The ultraviolet sterilization module 700 may include a bracket 750 for coupling the ultraviolet light PCB 720 and the cooler 730 to the housing of the massage apparatus. The bracket 750 may be coupled to the housing of the massage apparatus 100 by a screw.

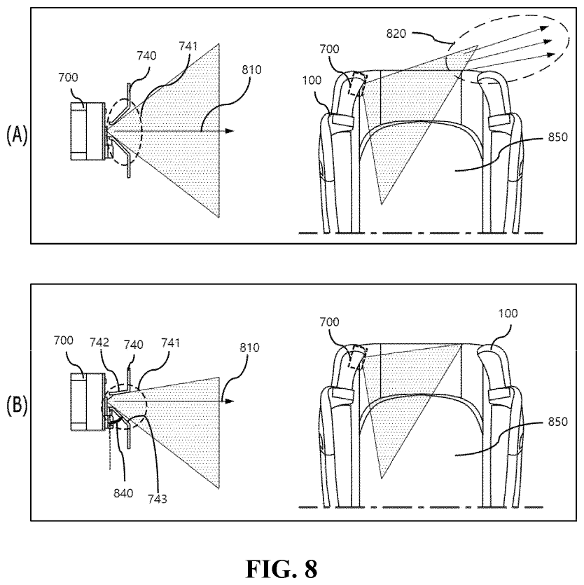

FIG. 8 is a diagram showing an ultraviolet sterilization module according to an embodiment of the present disclosure.

Referring to FIGS. 7 and 8, the housing 740 of the massage apparatus 100 may include a concave portion 741 curved into the housing 740 of the massage apparatus 100 at a position of the ultraviolet sterilization module 700. Referring to FIG. 7, the concave portion 741 may include four surfaces inclined with respect to a surface of the quartz 711 surface. However, the present disclosure is not limited thereto, and the concave portion 741 may include a plurality of surfaces inclined with respect to the surface of the quartz 711.

Referring to FIG. 8, the concave portion 741 may include a hole to allow an ultraviolet light source located inside the housing of the massage apparatus to emit ultraviolet light to the outside. The hole may be located in a direction of the ultraviolet sterilization module 700 in the concave portion 741.

The range of sterilization by ultraviolet light emitted from the ultraviolet light source may be determined based on the shape of the concave portion 741. Referring to (A) of FIG. 8, the concave portion 741 may be formed with an inner circumferential surface that expands as a distance from the ultraviolet sterilization module 700 increases. The concave portion 741 may include a plurality of surfaces inclined with respect to the surface of the quartz. The plurality of surfaces may be inclined at the same angle with respect to the surface of the quartz. Accordingly, ultraviolet light from an ultraviolet light source included in the ultraviolet sterilization module 700 may be gradually diffused to a surface perpendicular to a direction 810 while being emitted in the direction 810 perpendicular to the surface of the quartz. However, in this case, as shown in (A) of FIG. 8, some ultraviolet light 820 may be emitted to the outside of the massage apparatus 100. Therefore, ultraviolet light may be emitted to a user or an object outside the massage apparatus 100, and side effects may occur.

The massage apparatus 100 according to the present disclosure may adjust the shape of the concave portion 741 to prevent some ultraviolet light 820 from being emitted to the outside of the massage apparatus 100. For example, referring to (B) of FIG. 8, the concave portion 741 may be formed with an inner circumferential surface that expands as a distance from the ultraviolet sterilization module 700 increases. The concave portion 741 may include a plurality of surfaces inclined with respect to the surface of the quartz. The plurality of surfaces may be inclined at different angles to the surface of the quartz. For example, a first surface 742 included in the concave portion 741 may be almost perpendicular to the surface of the quartz, and an angle 840 between a second surface 743 included in the concave portion 741 and the surface of the quartz may be less than 90 degrees. Accordingly, by the shape of the concave portion 741, ultraviolet light may be deflected toward the second surface 743 and emitted. In addition, ultraviolet light may be emitted only to the massage apparatus 100 and may not be emitted to the outside of the massage apparatus 100. Accordingly, ultraviolet light may not be emitted to the user and objects around the massage apparatus 100.

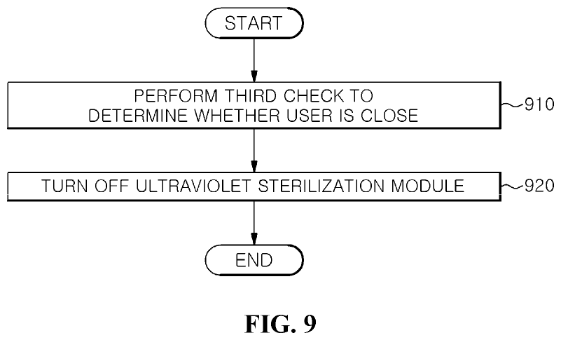

FIG. 9 is a flowchart for explaining an operation of a massage apparatus according to an embodiment of the present disclosure.

The massage apparatus 100 may perform operation 910 of performing a third check for determining whether the user is close to the massage apparatus 100 using at least one body sensor while being sterilized for a predetermined time using the ultraviolet sterilization module 700. If the first check and the second check are performed before ultraviolet light is emitted from the ultraviolet sterilization module 700, the third check may be performed while the massage apparatus 100 is being sterilized by the ultraviolet sterilization module 700. When performing the third check, the massage apparatus 100 may determine whether the user is close to the massage apparatus 100 using at least one of a seating sensor, a proximity sensor, and the massage module 2170. The massage apparatus 100 may perform the third check at a predetermined period.

When the user is close to the massage apparatus 100 in the third check, the massage apparatus 100 may perform operation 920 of turning off the ultraviolet sterilization module. In more detail, output of at least one body sensor may be whether the user is close to the massage apparatus 100. When output of at least one sensor indicates proximity of the user, the massage apparatus 100 may turn off the ultraviolet sterilization module. When output of at least one sensor does not indicate proximity of the user, the massage apparatus 100 may continuously perform ultraviolet sterilization.

Output of at least one body sensor may indicate a distance from the massage apparatus 100 to the user. For example, at least one body sensor may be an ultrasonic sensor or an infrared sensor. The massage apparatus 100 may adjust the intensity of ultraviolet light based on a distance measured by at least one body sensor. The massage apparatus 100 may adjust a sterilization time based on a distance measured by at least one body sensor. For example, the massage apparatus 100 may reduce the intensity of ultraviolet light as the measured distance is reduced. The massage apparatus 100 may increase the intensity of ultraviolet light as the measured distance is increased. The massage apparatus 100 may reduce an ultraviolet sterilization time as the measured distance is increased. The massage apparatus 100 may store a table in which the measured distance and the intensity of ultraviolet light correspond to each other. The massage apparatus 100 may store a table in which the measured distance and the ultraviolet sterilization time correspond to each other. The massage apparatus 100 may determine the ultraviolet sterilization time and the intensity of ultraviolet light based on the tables. Alternatively, the massage apparatus 100 may determine the ultraviolet sterilization time and the intensity of ultraviolet light based on a function. For example, the massage apparatus 100 may determine an ultraviolet sterilization time based on

T=c/1+d. Here, T is an ultraviolet sterilization time, 1 is a measured distance, and c and d are predetermined positive real numbers. In addition, T may correspond to a predetermined time of the present disclosure. The massage apparatus 100 may determine the intensity of ultraviolet light based on S=e*1+f. Here, S is the intensity of ultraviolet light, 1 is a measured distance, e is a predetermined positive real number, and d is a predetermined real number of 0 or more.

Through this process, the massage apparatus 100 may prepare for an unexpected access of the user during sterilization. Therefore, the massage apparatus 100 according to the present disclosure may promote user safety.

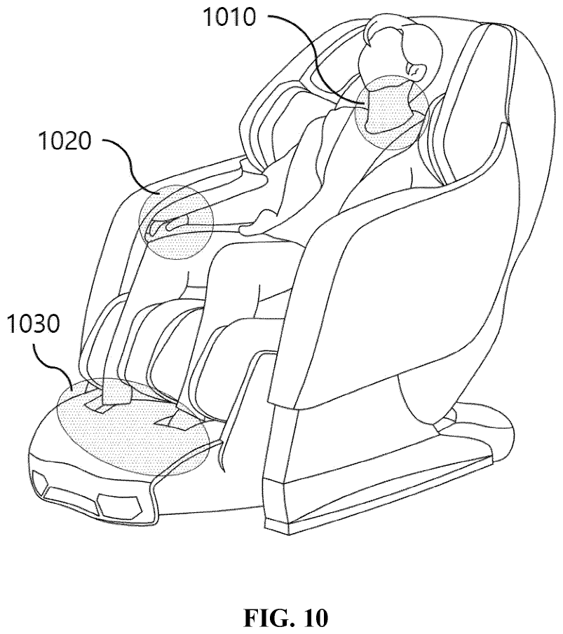

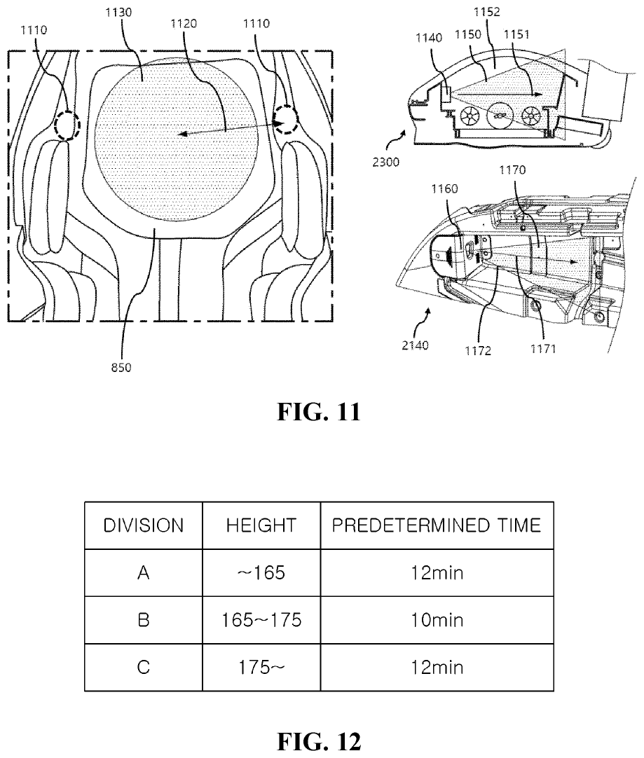

FIG. 10 is a diagram for explaining a sterilization range of a massage apparatus according to an embodiment of the present disclosure. FIG. 11 is a diagram for explaining a sterilization range of a massage apparatus according to an embodiment of the present disclosure.

Referring to FIG. 10, the target surface of the massage apparatus sterilized by the ultraviolet sterilization module may be position at a position corresponding to at least one of a neck 1010, a hand 1020, and a foot 1030 of the user. That is, the ultraviolet sterilization module 700 may emit ultraviolet light to the target surface of the massage apparatus 100 corresponding to the neck, hands, and feet of the user. The target surface of the massage apparatus 100 may be sterilized by ultraviolet light.

Although not shown in FIG. 10, the target surface of the massage apparatus 100 may be located at a position corresponding to buttocks of the user. That is, the ultraviolet sterilization module 700 may emit ultraviolet light to the target surface of the massage apparatus 100 corresponding to the buttocks of the user. The target surface of the massage apparatus 100 may be sterilized by ultraviolet light.

Referring to FIGS. 1 and 11, a housing 2111 may surround an upper end of an upper frame. The ultraviolet sterilization module 700 may be located inside the housing 2111.

Referring to FIG. 11, an ultraviolet sterilization module 1110 may be located inside a housing surrounding an upper end of the upper frame. The massage apparatus 100 may include the ultraviolet sterilization module 1110 for sterilizing a target surface 1130 that is in contact with the neck of the user. The ultraviolet sterilization module 1110 may be located inside the housing of the massage apparatus 100 near the ear of the user. For example, the ultraviolet sterilization module 1110 may be placed in various positions, such as at a speaker, at a shoulder massage part, or between the speaker and the shoulder massage part. The ultraviolet sterilization module 1110 may be located inside the housing of the arm massage part 2140. The ultraviolet sterilization module 1110 may emit ultraviolet light to a target surface including the seat pad or the back pad 850. For example, the ultraviolet sterilization module 1110 may emit ultraviolet light to the target surface 1130 located at a position corresponding to the neck or buttocks of the user.

A distance 1120 between the ultraviolet sterilization module 1110 and a target surface of the massage apparatus sterilized by the ultraviolet sterilization module may be 20 cm to 30 cm. If the ultraviolet sterilization module 1110 is excessively close to the target surface, a region of the target surface is excessively narrow, and thus an entire desired region may not be sterilized.

In addition, if the target surface is excessively far from the ultraviolet sterilization module 1110, the intensity of the ultraviolet light may decrease and it may take very long for actual sterilization to occur.

Referring to FIG. 11, the massage apparatus 100 may include an ultraviolet sterilization module 1140 for sterilizing a target surface that is in contact with the feet of the user. The leg massage part 2300 may include a foot massage part, and the ultraviolet sterilization module 1140 may be included in the foot massage part. The ultraviolet sterilization module 1140 may be located inside the housing of the leg massage part 2300 and located in front of a foot accommodation space 1152 of the housing of the leg massage part 2300.

The ultraviolet sterilization module 1140 may be included inside the foot massage part and thus may not be covered by a separate housing.

The ultraviolet sterilization module 1140 may be located in the housing of the massage apparatus near the toe of the user. The ultraviolet sterilization module 1140 may emit ultraviolet light toward the foot accommodation space 1152 from the front of the foot accommodation space.

The ultraviolet sterilization module 1140 may emit ultraviolet light toward a heel direction 1151 of the user. As described above, an irradiation region 1150 of ultraviolet light may be determined by the shape of the concave portion 741. In addition, the target surface of the massage apparatus 100 in the irradiation region 1150 of the ultraviolet light may be sterilized.

Referring to FIG. 11, the massage apparatus 100 may include an ultraviolet sterilization module 1160 for sterilizing a target surface in contact with the hand of the user. The ultraviolet sterilization module 1160 may be located inside the housing of the arm massage part 2140 and located in front of a hand/arm accommodation space 1172 of the housing of the arm massage part 2140. The ultraviolet sterilization module 1160 may be located inside the housing of the massage apparatus near the finger of the user. The ultraviolet sterilization module 1160 may emit ultraviolet light toward the hand/arm accommodation space 1172 from the front of the hand/arm accommodation space 1172. The ultraviolet sterilization module 1160 may emit ultraviolet light toward an elbow direction 1171 of the user. As described above, an irradiation region 1170 of ultraviolet light may be determined by the shape of the concave portion 741. In addition, a target surface of the massage apparatus 100 in the irradiation region 1170 of ultraviolet light may be sterilized.

The massage apparatus 100 may include at least one of a dust sensor, a temperature sensor, or a humidity sensor. The massage apparatus 100 may include at least one of a dust sensor, a temperature sensor, and a humidity sensor in each of a neck 1010, a hand 1020, and a foot 1030.

The massage apparatus 100 may perform ultraviolet sterilization on only some parts based on sensor values. That is, the massage apparatus 100 may sterilize an area with high dust concentration, high temperature, or high humidity.

For example, the massage apparatus 100 may obtain measurement values from sensors located on the neck 1010, the hand 1020, and the foot 1030. The measurement values may be periodically obtained while a previous user uses the massage apparatus 100. The measurement value of the massage apparatus 100 may be one of dust concentration, temperature, and humidity. The massage apparatus 100 may obtain a maximum value or an average value among periodically obtained measurement values as a representative value. The massage apparatus 100 may perform ultraviolet sterilization on a corresponding region when the representative value is greater than or equal to a threshold value. For example, the massage apparatus 100 may perform ultraviolet sterilization on an area where a representative value of dust concentration is higher than a threshold value. In addition, the massage apparatus 100 may perform ultraviolet sterilization on an area where a representative value of temperature is higher than a threshold value. The massage apparatus 100 may perform ultraviolet sterilization on an area where a representative value of humidity is higher than a threshold value. As such, energy consumed by the ultraviolet sterilization module may be saved by sterilizing some parts instead of performing ultraviolet sterilization for all parts.

FIG. 12 is a diagram for explaining a method of determining a sterilization time according to an embodiment of the present disclosure.

The controller 300 may perform an operation of estimating the position of the shoulder of the previous user. The controller 300 may perform an operation of estimating the position of the shoulder using the massage module 2170. The controller 300 may perform an operation of determining a predetermined time based on the estimated position of the shoulder and a pre-stored table. The controller 300 may determine the predetermined time based on a comparison between the estimated position of the shoulder and a pre-stored reference value. The reference value may be the position of a reference shoulder. For example, the massage apparatus 100 may store a table in which the estimated position of the shoulder and a predetermined time correspond to each other. In the table, a range of estimated positions of the shoulder may correspond to a predetermined time. The massage apparatus 100 may select a predetermined time corresponding to the estimated position of the shoulder from the table. The predetermined time may be selected based on the table to become longer as the position of the shoulder is further away from the position of the reference shoulder. The position of the reference shoulder may be a predetermined position of the shoulder and may be an average position of the shoulder of the user. The massage apparatus 100 may operate the ultraviolet sterilization module 700 for the selected predetermined time period.

In more detail, the massage apparatus 100 may perform an operation of estimating the height of the previous user using at least one height estimation sensor. For example, the massage apparatus may estimate the height of a user using the massage apparatus 100 immediately before sterilization using the massage module 2170. For example, the massage apparatus 100 may move the massage module 2170 to the top. The massage apparatus 100 may lower the massage module 2170 slowly. When a user is sitting in the massage apparatus 100, the massage module 2170 may be positioned on the shoulder of the user while descending. At this time, a load of the actuator (driver) for moving the massage module 2170 may increase due to resistance of the massage module 2170 and the shoulder of the user. The massage apparatus 100 may acquire information about the position of the shoulder of the previous user by recognizing a point where the load of the actuator is high as the position of the shoulder of the user. The massage apparatus 100 may store information about the position of the shoulder of the previous user.

The massage apparatus 100 may estimate the height of the user based on the information on the position of the shoulder of the previous user. For example, the massage apparatus 100 may pre-store a table in which information on the position of the shoulder and the height of the user correspond to each other. The massage apparatus 100 may acquire the estimated height of the user corresponding to the information on the position of the shoulder of the user.

The massage apparatus 100 may store the estimated height.

According to various embodiments of the present disclosure, the massage apparatus 100 may not estimate a height. The massage apparatus 100 may not estimate the height of the user based on information on the position of the shoulder of the previous user. The massage apparatus 100 may store a table in which information on the position of the shoulder of the previous user corresponds to a predetermined time. In the table, a range of the information on the position of the shoulder of the previous user may correspond to a predetermined time. The predetermined time may be selected based on the table to become longer as the position of the shoulder is further away from the position of the reference shoulder. The position of the reference shoulder may be a predetermined position of the shoulder and may be an average position of the shoulder of the user. The massage apparatus 100 may select a predetermined time from the table based on the acquired information on the position of the shoulder of the previous user. The massage apparatus 100 may operate the ultraviolet sterilization module 700 to sterilize a target surface for the selected predetermined time.

However, the present disclosure is not limited thereto, and the massage apparatus 100 may receive the height of the user from the user. The massage apparatus may store the received height.

The massage apparatus 100 may perform an operation of determining a predetermined time based on an absolute value of a difference between the estimated height and the previously stored reference height. The pre-stored reference height may be a value previously determined by a manufacturer. The massage apparatus 100 may obtain the absolute value by subtracting the previously stored reference height from the height of the previous user. The massage apparatus 100 may determine a predetermined time for sterilization based on the absolute value. In the above, the operation of determining the predetermined time using the absolute value of the difference between the estimated height and the pre-stored reference height has been described, but the present disclosure is not limited thereto. The massage apparatus 100 may obtain an absolute value of a difference between the estimated position of the shoulder and the previously stored reference position of the shoulder. The massage apparatus 100 may perform an operation of determining a predetermined time based on the absolute value related to the position of the shoulder.

For example, the massage apparatus 100 may store a table in which the absolute value corresponds to the predetermined time. In the table, the absolute value and the predetermined time may be proportionally corresponded. The massage apparatus 100 may select a predetermined time corresponding to the absolute value in the table.

The massage apparatus 100 may obtain a predetermined time from the absolute value based on a predetermined function. The predetermined function may be T=a*A+b. Here, T is a predetermined time, A is an absolute value, a is a predetermined positive real number, and b may be a predetermined positive real number. That is, the absolute value may be proportional to a predetermined time.

According to the present disclosure, T may be determined based on the absolute value and a distance between the user and the massage apparatus. T may be determined according to a*A+c/1+b+d. As described above, here, T may be an ultraviolet sterilization time, 1 is a measured distance, and c and d are predetermined positive real numbers. In addition, A is an absolute value, a is a predetermined positive real number, and b may be a predetermined positive real number.

However, the present disclosure is not limited thereto, and the massage apparatus 100 may pre-store a table in which the height of the user corresponds to a predetermined time. This may be the same as shown in FIG. 12. The massage apparatus 100 may acquire a predetermined time corresponding to the height of the user from the table. For example, when the height of the user is equal to or less than 165 cm, the predetermined time may be 12 minutes. When the height of the user is greater than 165 cm and equal to or less than 175 cm, the predetermined time may be 10 minutes. When the height of the user is greater than 175 cm, the predetermined time may be 12 minutes. However, the present disclosure is not limited thereto, and the massage apparatus 100 may use various tables.

The massage apparatus 100 may determine a sterilization time based on a measurement value of a dust sensor, a massage time of a previous user, the current temperature, and the current humidity. For example, the massage apparatus 100 may include a dust sensor, and may acquire a concentration value of dust from the dust sensor before ultraviolet sterilization starts. When the concentration value of dust is higher than a predetermined threshold value, the massage apparatus 100 may determine an ultraviolet sterilization time by adding an additional time to the predetermined time. The massage apparatus 100 may acquire the massage of the previous user from a memory. When the massage time of the previous user is higher than a predetermined threshold value, the massage apparatus 100 may determine the ultraviolet sterilization time by adding an additional time to the predetermined time. The massage apparatus 100 may acquire the current temperature or the current humidity from the sensor. When the current temperature or the current humidity is higher than a predetermined threshold value, the massage apparatus 100 may determine an ultraviolet sterilization time by adding an additional time to the predetermined time.

The massage apparatus 100 may accumulate and store a sterilization time point and time period after sterilization is performed. The massage apparatus 100 may output the sterilization time point and time period through an output interface based on an input of the user. If desired, the user may be capable of checking the time point and time period when sterilization is performed, and thus it may be easy to determine whether sterilization is currently required for the massage apparatus 100. In addition, the user may input a signal indicating that sterilization is supposed to be performed on the massage apparatus 100 if necessary. The massage apparatus 100 may perform ultraviolet sterilization based on an input of the user.

The controller 300 may determine the predetermined time based on a usage time of the massage apparatus 100, a sitting time, or a time of receiving massage of the previous user. The usage time of the massage apparatus 100 may mean a time when a user uses various services provided by the massage apparatus 100. Recently, the massage apparatus 100 not only provides massage, but also provides music listening and video viewing services. The usage time of the massage apparatus 100 may mean a time the user uses such a service. The time the user only sits in the massage apparatus 100 without receiving service from the massage apparatus 100 may not be counted. The sitting time of the previous user may mean a time the user is sitting on the massage apparatus 100. The massage apparatus 100 may determine the sitting time of the previous user using the seating sensor. For example, the massage apparatus 100 may determine a time from when the seating sensor detects sitting of the previous user to a moment when the seating sensor detects leaving as the seating time. The sitting time may include a state in which the user is simply sitting or lying on the massage apparatus 100. The time when the massage is provided to the previous user may be a time when the massage apparatus 100 provides massage to the user.

The massage apparatus 100 may determine a predetermined time in direct proportion to the usage time, the sitting time, or the time of receiving massage. The massage apparatus 100 can determine a predetermined time based on a predetermined function or table. The function may be T=k*t+m. Here, T is a predetermined time, k is a predetermined amount of real number, m is a predetermined real number, and t is a usage time, a sitting time, or a time of receiving massage. The table may be a table in which the usage time, the sitting time, or the time of receiving massage corresponds to a predetermined time.

The massage apparatus 100 may accumulate and store usage times, sitting times, or times of receiving massage of previous users. The massage apparatus 100 may determine a predetermined time in direct proportion to the accumulated usage times, sitting times, or times of receiving massage of the previous users before operating the ultraviolet sterilization module. The massage apparatus 100 may sterilize the target surface of the massage apparatus 100 by operating the ultraviolet sterilization module based on a predetermined time.

While this disclosure has been particularly shown and described with reference to exemplary embodiments thereof, it will be understood by those skilled in the art that various changes in form and details may be made therein without departing from the spirit and scope of the present disclosure as defined by the appended claims. The exemplary embodiments need to be considered in descriptive sense only and not for purposes of limitation. Therefore, the scope of the present disclosure is defined not by the detailed description of the present disclosure but by the appended claims, and all differences within the scope will be construed as being included in the present disclosure.

The embodiments of the present disclosure can be written as computer programs and can be implemented in general-use digital computers that execute the programs using a computer readable recording medium. Examples of the computer readable recording medium include magnetic storage media (e.g., ROM, floppy disks, hard disks, etc.), and storage media such as optical recording media (e.g., CD-ROMs, or DVDs).

1. A massage apparatus comprising:
a main frame defining a frame of the massage apparatus and including an upper frame and a base frame;
a seat pad or a back pad formed on the upper frame and increasing a seating feel of a user;
an arm massage part formed on both sides of the main frame and providing massage to an arm of the user;
an ultraviolet sterilization module for sterilizing and disinfecting at least some of the seat pad, the back pad, or the arm massage part;
a controller for controlling an operation of the massage apparatus; and
at least one body sensor for performing a first check to determine whether the user is close to the massage apparatus, wherein, when the user is not close to the massage apparatus, the controller sterilizes the massage apparatus for a predetermined time using the ultraviolet sterilization module.

2. The massage apparatus of claim 1, wherein the ultraviolet sterilization module includes:
a light source cover for transmitting ultraviolet light and preventing a foreign substance from entering an ultraviolet light source;
an ultraviolet light PCB including the ultraviolet light source and a circuit for driving the ultraviolet light source;
a cooler for cooling the ultraviolet light PCB; and
a bracket for coupling the ultraviolet light PCB and the cooler to a housing of the massage apparatus.

3. The massage apparatus of claim 2, wherein the light source cover includes quartz for transmitting ultraviolet light having a wavelength of 100 nm or more and 280 nm or less.

4. The massage apparatus of claim 2, wherein the housing of the massage apparatus includes a concave portion curved into the housing of the massage apparatus at a position of the ultraviolet sterilization module,
the concave portion includes hole to allow the ultraviolet light source located inside the housing of the massage apparatus to emit ultraviolet light to an outside, and
a range of sterilization by ultraviolet light emitted from the ultraviolet light source is determined based on a shape of the concave portion.

5. The massage apparatus of claim 1, wherein, while the massage apparatus is sterilized for a predetermined time using the ultraviolet sterilization module, a signal indicating that the user is prevented from approaching a vicinity of the massage apparatus is output through an output interface.

6. The massage apparatus of claim 4, wherein, while the massage apparatus is sterilized for a predetermined time using the ultraviolet sterilization module, the controller performs a third check to determine whether the user is close to the massage apparatus using the at least one body sensor, and when the user is close to the massage apparatus in the third check, the controller controls and turns off the ultraviolet sterilization module.

7. The massage apparatus of claim 1, wherein a target surface of the massage apparatus, sterilized by the ultraviolet sterilization module, is located at a position at which the user and the massage apparatus are in contact with each other.

8. The massage apparatus of claim 7, wherein the ultraviolet sterilization module is located inside a housing surrounding an upper end of the upper frame and emits ultraviolet light to the target surface including the seat pad or the back pad.

9. The massage apparatus of claim 7, wherein the ultraviolet sterilization module is located inside a housing of the arm massage part, is disposed in front of a hand/arm accommodation space of the housing of the arm massage part, and emits ultraviolet light toward the hand/arm accommodation space from a front of the hand/arm accommodation space.

10. The massage apparatus of claim 7, further comprising:
a leg massage part formed in front of the main frame and providing massage to a leg of the user,
wherein the ultraviolet sterilization module is located inside a housing of the leg massage part, is disposed in front of a foot accommodation space of the housing of the leg massage part, and emits ultraviolet light toward the foot accommodation space from a front of the foot accommodation space.

11. The massage apparatus of claim 1, wherein the controller estimates a position of a shoulder of a previous user using the massage module, and determines the predetermined time based on the estimated position of the shoulder and a pre-stored table.

12. The massage apparatus of claim 1, wherein the controller determines the predetermined time based on a usage time, a sitting time, or a time of receiving massage of the massage apparatus for a previous user.
